(19) United States
(12) Patent Application Publication
(10) Pub. No.: US 2013/0303780 A1
(43) Pub. Date: Nov. 14, 2013

(54) PROCESS FOR THE PREPARATION OF 2-ARYLTHIAZOLE DERIVATIVES

(71) Applicants: Siva Rama Prasad Vellanki, Hyderabad (IN); Arabinda Sahu, Hyderabad (IN); Satyanarayana Raavi, Hyderabad (IN); Ravi Nuchu, Hyderabad (IN); Ramesh Dandala, Hyderabad (IN)

(72) Inventors: Siva Rama Prasad Vellanki, Hyderabad (IN); Arabinda Sahu, Hyderabad (IN); Satyanarayana Raavi, Hyderabad (IN); Ravi Nuchu, Hyderabad (IN); Ramesh Dandala, Hyderabad (IN)

(21) Appl. No.: 13/883,843

(22) PCT Filed: Nov. 3, 2011
(86) PCT No.: PCT/IN2011/000761

§ 371 Date: May 7, 2013

(30) Foreign Application Priority Data
Nov. 8, 2010 (IN) 3312/CHE/2010

(51) Int. Cl. C07D 277/56

(52) U.S. Cl. CPC C07D 277/56
USPC 548/201

(57) ABSTRACT

The present invention relates an improved process for the preparation of 2-arylthiazole derivatives which are intermediates of Febuxostat and further conversion to Febuxostat or pharmaceutically acceptable salts thereof.

This application claims priority to Indian patent application No. 3312/CHE/2010 filed on Nov. 4, 2010.

### FIELD OF THE INVENTION

The present invention relates an improved process for the preparation of 2-Arylthiazole derivatives which are intermediates of Febuxostat and further conversion to Febuxostat or pharmaceutically acceptable salts thereof.

### BACKGROUND OF THE INVENTION

2-Arylthiazole derivatives are used as xanthine oxidase inhibitors for use in the treatment of hyperuricemia and gout.

Febuxostat, 2-(3-cyano-4-isobutyloxyphenyl)-4-methyl-5-thiazolecarboxylic acid of Formula-I is an example of 2-arylthiazole derivatives used as xanthine oxidase inhibitors for use in the treatment of hyperuricemia and gout.

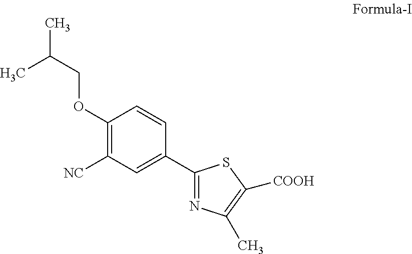 [SMILES: Cc1nc(-c2ccc(OCC(C)C)c(C#N)c2)sc1C(=O)O]

Febuxostat of Formula-I is approved by USFDA for the treatment of hyperuricemia in patients with gout under the brand name of ULORIC. ULORIC is recommended at 40 mg or 80 mg once daily.

Febuxostat and its pharmaceutically acceptable salts were first disclosed in United States patent publication U.S. Pat. No. 5,614,520. This patent also discloses process for the preparation of Febuxostat.

Japan patent publications JP 2834971 and JP 3202607 discloses process for the preparation of Febuxostat through the cyano intermediate compound of Formula-II.

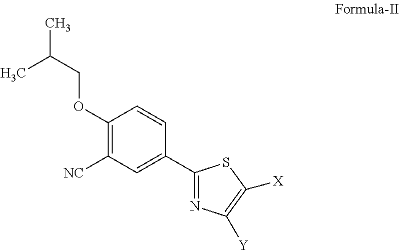

wherein R is C₁-C₁₀ alkyl or arylalkyl, X and Y, independently of each other, represents a hydrogen atom, C₁-C₄ alkyl, C₁-C₈ carboxyl, C₁-C₈ alkoxycarbonyl or aryloxycarbonyl group. The process disclosed in these patents is given in scheme-1

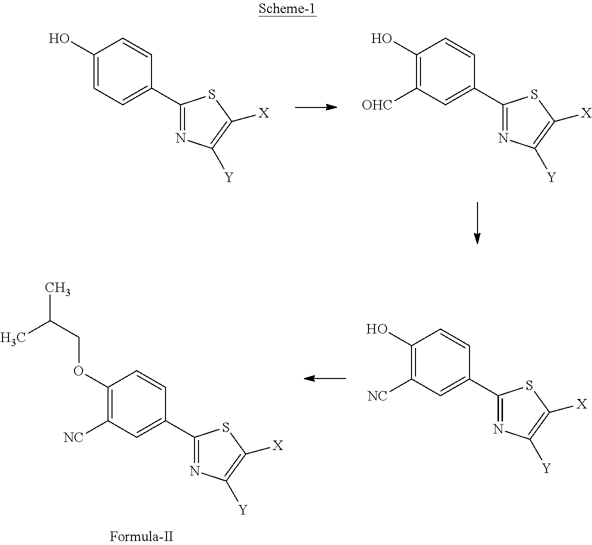

The above process involves more synthetic steps in the preparation of compound of Formula-II and yield obtained by the above mentioned processes is less.

Thus the present invention provides process for the preparation of compound of Formula-II with high yield and it is further converted into Febuxostat or its pharmaceutically acceptable salts.

### OBJECT AND SUMMARY OF THE INVENTION

The principle object of the present invention is to provide an improved process for the preparation of compound of Formula-II through compound of Formula-III.

Another object of the present invention is to provide further conversion of compound of Formula-II into Febuxostat and its pharmaceutically acceptable salts.

In one aspect the present invention provides, an improved process for the preparation of compound of Formula-II comprising the steps of:
a) reacting the compound of Formula-III with hydroxylamine hydrochloride in an organic solvent;

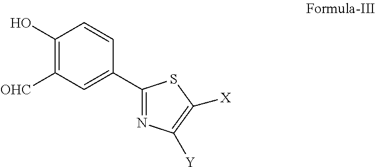

b) adding acyl halides or sulfonyl chlorides to the reaction mixture;
c) adding a base and isobutyl halide;
d) isolating the compound of Formula-II

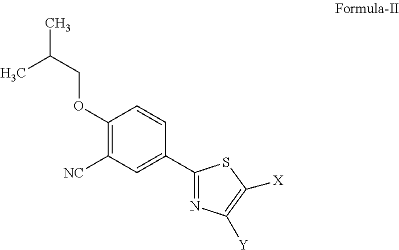

### DETAILED DESCRIPTION OF THE INVENTION

The present invention relates to an improved process for the preparation of Alkyl-2-(3-cyano-4-alkoxy phenyl)-thaizole compound of Formula-II through Alkyl-2-(3-formyl-4-hydroxy phenyl)-thaizole compound of Formula-III. The present invention further relates to the conversion of compound of Formula-II to Febuxostat.

The main aspect of the present invention provides an improved process for the preparation of compound of Formula-II comprising the steps of:
a) reacting the compound of Formula-III with hydroxylamine hydrochloride in an organic solvent;

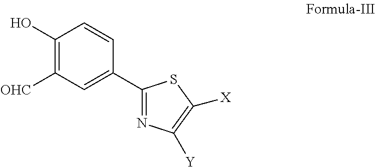

wherein X and Y, independently of each other, represents a hydrogen atom, C₁-C₄ alkyl, C₁-C₈ carboxyl, C₁-C₈ alkoxycarbonyl or aryloxycarbonyl group.
b) adding acyl halides or sulfonyl chlorides to the reaction mixture;
c) optionally isolating compound of Formula-IV;

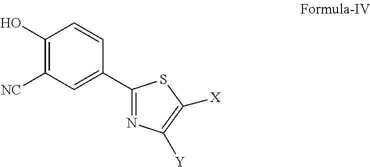

d) reacting with isobutyl halide in presence of base;
e) isolating the compound of Formula-II

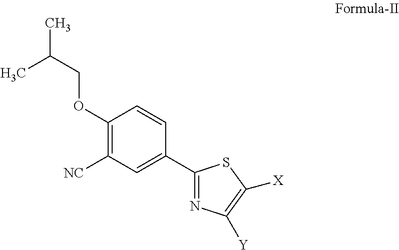

In one embodiment, the organic solvent used in the step-a, is selected from polar aprotic solvents such as dimethyl sulfoxide (DMSO), Dimethylacetamide (DMA), Acetonitrile (ACN) or dimethyl formamide (DMF).

In one more embodiment, acyl halide used in the step-b, is selected from acetyl bromide or acetyl chloride.

In one more embodiment, sulfonyl chlorides used in the step-b, is selected from methane sulfonyl chloride or para toluene sulfonyl chloride.

In one more embodiment, base used in the step-d is selected from alkali metal carbonates, such as potassium carbonate or sodium carbonate, preferably potassium carbonate.

In one more embodiment, isobutyl halide used in step-d is selected isobutyl chloride or isobutyl bromide.

In another embodiment, the process of step-a to step-d is carried out in a single step without isolating the intermediates.

In another embodiment, the compound of Formula-II prepared by the above process is

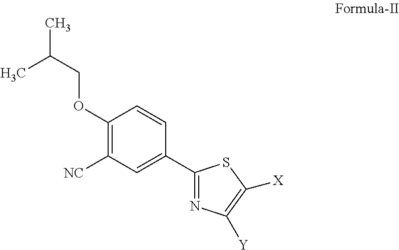

wherein X is C₁-C₈ alkoxycarbonyl or arylalkoxycarbonyl and Y is methyl.

In one more embodiment, the compound of Formula-II is further converted to Febuxostat by hydrolysis. The hydrolysis of compound of Formula-II can be carried out in presence of aqueous Methanol, aqueous Ethanol, aqueous Isopropanol, aqueous Acetone and aqueous Acetonitrile. The hydrolysis also carried out using water with mixture of solvents like Ethanol and Tetrahydrofuran; Methanol and Tetrahydrofuran; Acetone and Tetrahydrofuran; Acetonitrile and Tetrahydrofuran; Isopropanol and Tetrahydrofuran.

As per the present invention, hydroxylamine hydrochloride is added to compound of Formula-III in presence of a polar aprotic solvent like DMSO, DMA, ACN or DMF. To this reaction mixture acetyl halides or sulfonyl chlorides are added and temperature raised to 70-80° C. Acetyl halides are selected from acetyl bromide or acetyl chloride. Sulfonyl chlorides are selected from methane sulfonyl chloride or para toluene sulfonyl chloride. To this reaction mixture a base selected from alkali metal carbonates like potassium carbonate or sodium carbonate, preferably potassium carbonate and alkyl halide selected from isobutyl bromide is successively added. The reaction mass is washed with water and compound of Formula-II is isolated.

In one embodiment the present invention provides, process for the preparation of Febuxostat comprising the steps of:
a) reacting the compound of Formula-III(a) with hydroxylamine hydrochloride in presence of organic solvent;

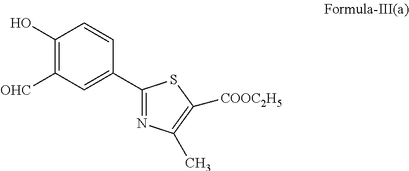

b) adding acyl halides or sulfonyl chlorides to the reaction mixture;
c) optionally isolating compound of Formula-IV(a)

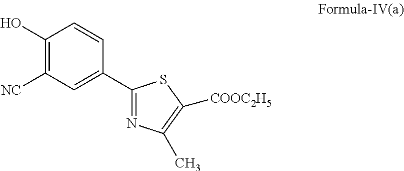

d) reacting with isobutyl bromide in presence of base;
e) isolating the compound of Formula-II(a); and

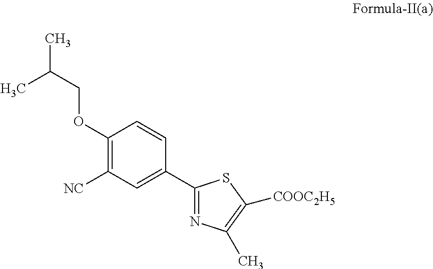

f) hydrolyzing the compound of Formula-II(a) to get Febuxostat.

The following examples are provided to illustrate the process of the present invention. They, are however, not intended to limiting the scope of the present invention in any way and several variants of these examples would be evident to person ordinarily skilled in the art.

### EXPERIMENTAL PROCEDURE

### Example-1

### Preparation of Ethyl-2-(3-cyano-4-isobutoxy phenyl)-4-methyl thiozole-5-carboxylate

A mixture of 10.0 g of Ethyl-2-(3-formyl-4-hydroxy phenyl)-4-methyl thiozole-5-carboxylate and 2.85 g of hydroxylamine hydrochloride were stirred for 30 minutes in 40 g of Dimethyl sulfoxide. To this reaction mixture 3.3 grams of acetyl chloride was added and stirred at 70-80° C. for 2-3 hours. Reaction mass was cooled to room temperature and to this 19 g of potassium carbonate and 19 g of isobutyl bromide was added successively. The reaction mass was stirred for 5 hours at 70-80° C. Reaction mass was diluted with 200 ml of purified water. The reaction mass was filtered and washed with purified water to give 10.0 g of Ethyl-2-(3-cyano-4-isobutoxy phenyl)-4-methyl thiozole-5-carboxylate (yield 84.0%)

### Example-2

### Preparation of Ethyl-2-(3-cyano-4-hydroxyphenyl)-4-methyl thiozole-5-carboxylate

A mixture of 10.0 g of Ethyl-2-(3-formyl-4-hydroxy phenyl)-4-methyl thiozole-5-carboxylate and 2.85 g of hydroxylamine hydrochloride were stirred for 30 minutes in 30 g of Dimethylformamide. To this reaction mixture 3.3 grams of acetyl chloride was added and stirred at 90° C. for 2-3 hours. Reaction mass was cooled to room temperature and diluted with 100 ml of water and stir for 2 hours. The reaction mass was filtered and washed with purified water to give 10.0 g of Ethyl-2-(3-cyano-4-hydroxy phenyl)-4-methyl thiozole-5-carboxylate (yield 99.0%).

Example-3

### Preparation of Ethyl 2-(3-cyano-4-isobutoxy phenyl)-4-methyl thiozole-5-carboxylate

A mixture of 10.0 g of Ethyl-2-(3-cyano-4-hydroxy phenyl)-4-methyl thiozole-5-carboxylate, 30 g of NMP, 9.6 g of potassium carbonate and 7.2 g of isobutyl bromide were stirred for 3 hours at 90° C. Reaction mass was diluted with 100 ml of purified water. The reaction mass was filtered and washed with purified water and ethanol to give 10.5 g of Ethyl-2-(3-cyano-4-isobutoxy phenyl)-4-methyl thiozole-5-carboxylate (yield 88.0%).

### Example-4

### Preparation of 2-(3-cyano-4-isobutoxy phenyl)-4-methyl thiozole-5-carboxylic acid

A mixture of 10.0 g of Ethyl-2-(3-cyano-4-isobutoxy phenyl)-4-methyl thiozole-5-carboxylate, 2.0 g of sodium hydroxide was heated at 45-60° C. in 75 ml of aqueous methanol for 1 hour. Reaction mass was cooled to ambient temperature and pH adjusted to 2.0 to 2.5 with dilute hydrochloric acid and precipitated crystal was collected by filtration to give 8.8 g of 2-(3-cyano-4-isobutoxy phenyl)-4-methyl thiozole-5-carboxylic acid (yield 95.8%).

### Example-5-13

### Preparation of 2-(3-cyano-4-isobutoxy phenyl)-4-methyl thiozole-5-carboxylic acid

The above compound was prepared by following the procedure as disclosed in Example-4, using the below listed solvents instead of aqueous methanol.

| | |
|---|---|
| Example 5 | aqueous Ethanol |
| Example 6 | aqueous Isopropanol |
| Example 7 | aqueous Acetone |
| Example 8 | aqueous Acetonitrile |
| Example 9 | Water and mixture of Methanol + Tetrahydrofuran |
| Example 10 | Water and mixture of Ethanol + Tetrahydrofuran |
| Example 11 | Water and mixture of Acetone + Tetrahydrofuran |
| Example 12 | Water and mixture of Acetonitrile + Tetrahydrofuran |
| Example 13 | Water and mixture of Isopropanol + Tetrahydrofuran |

### Example-14

### Preparation of pure 2-(3-cyano-4-isobutoxy phenyl)-4-methyl thiozole-5-carboxylic acid

10.0 g of 2-(3-cyano-4-isobutoxy phenyl)-4-methyl thiozole-5-carboxylic acid was dissolved in 100 ml of ethanol at reflux temperature. After dissolution reaction mass was cooled and precipitated crystal was collected by filtration to give 9.6 g of pure 2-(3-cyano-4-isobutoxy phenyl)-4-methyl thiozole-5-carboxylic acid (yield 96%).

1. A process for the preparation of a compound of Formula-II 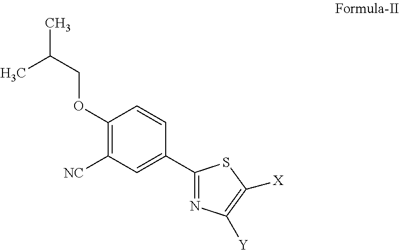
wherein X is C₁-C₈ alkoxycarbonyl or arylalkoxycarbonyl and Y is methyl, comprising the steps of:
a) reacting a compound of Formula-III with hydroxylamine hydrochloride in an organic solvent; 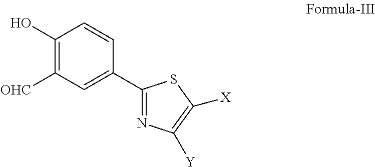
b) adding an acyl halides or a sulfonyl chlorides to the reaction mixture to yield a compound of Formula-IV;
c) optionally isolating the compound of Formula-IV; 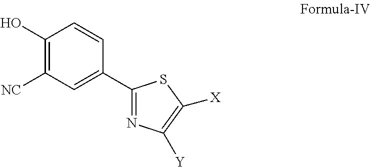
d) reacting the compound of Formula-IV with an isobutyl halide in the presence of a base to yield a compound of Formula-II; and
e) isolating the compound of Formula-II.

2. The process according to claim 1, wherein the organic solvent is selected from the group consisting of dimethyl sulfoxide, dimethylacetamide, dimethyl formamide and acetonitrile.

3. The process according to claim 1, wherein the acyl halide is selected from the group consisting of acetyl bromide and acetyl chloride.

4. The process according to claim 1, wherein the sulfonyl chloride is selected from the group consisting of methane sulfonyl chloride and para-toluene sulfonyl chloride.

5. The process according to claim 1, wherein the base is an alkali metal carbonate.

6. The process according to claim 1, wherein the compound of formula-II is further hydrolyzed to Febuxostat or a pharmaceutically acceptable salts thereof.

7. The process according to claim 6, wherein the compound of formula-II is hydrolyzed by using aqueous ethanol, aqueous methanol, aqueous acetone, aqueous acetonitrile or aqueous isopropyl alcohol.

8. A process for the preparation of Febuxostat comprising the hydrolyzation of a compound of Formula-II 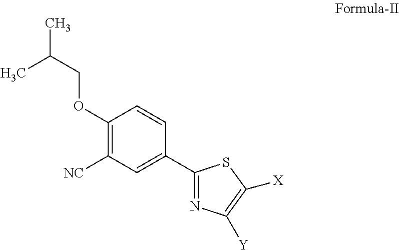
wherein X is C₁-C₈ alkoxycarbonyl or arylalkoxycarbonyl and Y is methyl, in the presence of aqueous acetone or aqueous acetonitrile.

10. (canceled)

11. A process for the preparation of Febuxostat comprising the steps of:
a) reacting a compound of Formula-III(a) with hydroxylamine hydrochloride in the presence of an organic solvent; 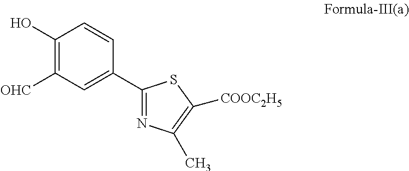
b) adding an acyl halide or a sulfonyl chloride to the reaction mixture to yield a compound of Formula-IV(a);
c) optionally isolating the compound of Formula-IV(a) 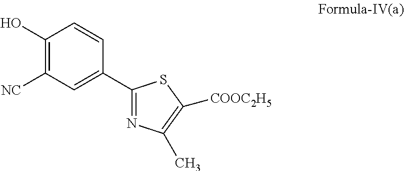
d) reacting the compound of Formula-IV(a) with isobutyl bromide in the presence of a base to yield a compound of Formula-II(a);
e) isolating the compound of Formula-II(a); and 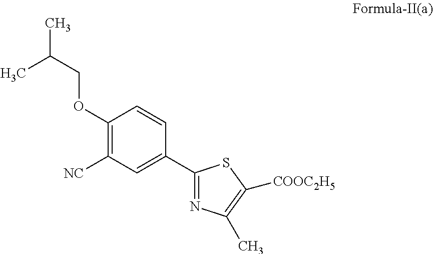
f) hydrolyzing the compound of Formula-II(a) to get Febuxostat.

12. The process according to claim 5, wherein the alkali metal carbonate is potassium carbonate or sodium carbonate.
